SEQUENCE LISTING (1 sequence)

SEQ ID NO: 1 - protein, 21 aa, Clostridium tetani
FNNFTVSFWLRVPKVSASHLE

(19) United States
(12) Patent Application Publication
(10) Pub. No.: US 2009/0098090 A1
(43) Pub. Date: Apr. 16, 2009

(54) VITRO IMMUNIZATION

(75) Inventors: Derek Nigel John Hart, Queensland (AU); Cameron John Turtle, Queensland (AU)

Correspondence Address: KNOBBE MARTENS OLSON & BEAR LLP, 2040 MAIN STREET, FOURTEENTH FLOOR, IRVINE, CA, 92614, US

(73) Assignee: THE CORPORATION OF THE TRUSTEES OF THE ORDER OF THE SISTERS OF MERCY IN QUEENSLAND

(21) Appl. No.: 10/538,393

(22) PCT Filed: Dec. 9, 2003
(86) PCT No.: PCT/AU03/01647

§ 371 Date: Nov. 13, 2006

(30) Foreign Application Priority Data
Dec. 9, 2002 (AU) 2002953238

(51) Int. Cl. A61K 35/14; C12N 5/08

(52) U.S. Cl. 424/93.7; 435/372

(57) ABSTRACT

The present invention relates generally to a method of generating lymphocytes specific for particular antigens. More particularly, the present invention provides a method for generating antigen-reactive T- cells and even more particularly cytotoxic (CD8+) T-cells in 10 vitro specific for antigens such as peptide antigens. The method of the present invention enables in vitro T-cell priming for particular antigens such as antigens on cancer cells, pathogenic cells, viruses or cells infected with viruses. The present invention is useful in identifying particularly immunogenic antigens for immunotherapy. Furthermore, as a consequence, the present invention is useful in avoiding the need for expensive and time 15 consuming clinical trials. The present invention further provides a method for the treatment or prophylaxis of a disease or condition in a subject by generating T-cells reactive to an antigenic molecule and administering an effective amount of antigen-reactive T-cells to the subject or other compatible host. Furthermore, the present invention permits the generation of dendritic cell/T-cell populations for use in cellular immunotherapy.

### BACKGROUND OF THE INVENTION

1. Field of the Invention

The present invention relates generally to a method of generating lymphocytes specific for particular antigens. More particularly, the present invention provides a method for generating antigen-reactive T-cells and even more particularly cytotoxic (CD8⁺) T-cells in vitro specific for antigens such as peptide antigens. The method of the present invention enables in vitro T-cell priming for particular antigens such as antigens on cancer cells, pathogenic cells, viruses or cells infected with viruses. The present invention is useful in identifying particularly immunogenic antigens for immunotherapy. Furthermore, as a consequence, the present invention is useful in avoiding the need for expensive and time consuming clinical trials. The present invention further provides a method for the treatment or prophylaxis of a disease or condition in a subject by generating T-cells reactive to an antigenic molecule and administering an effective amount of antigen-reactive T-cells to the subject or other compatible host. Furthermore, the present invention permits the generation of dendritic cell/T-cell populations for use in cellular immunotherapy.

2. Description of the Prior Art

Bibliographic details of the publications referred to by author in this specification are collected at the end of the description.

Reference to any prior art in this specification is not, and should not be taken as, an acknowledgment or any form of suggestion that this prior art forms part of the common general knowledge in any country.

The immune system is a protective mechanism which operates to varying extents in all higher vertebrate animals. The immune system controls the immune response to a foreign body (i.e. an antigen) present on, for example, a pathogen or even on a cancer cell. An autoimmune response occurs when the antigen is a “self” molecule, i.e. present in the particular vertebrate animal mounting the response. Such an immune response can lead to, for example, autoimmune diabetes.

Immunization, whether following an infection or as a result of human intervention, aims to induce an early protective response. Artificial immunization has been very successful in preventing infectious diseases such as polio, tetanus and diphtheria.

Cells of the immune system arise from pluripotent stem cells through two main lines of differentiation:
(a) the lymphoid lineage producing lymphocytes (T-cells, B-cells, natural killer cells, dendritic cells); and
(b) the myeloid lineage (monocytes, macrophages and neutrophils) as well as accessory cells including dendritic cells, platelets and mast cells.

In the circulatory system and secondary lymphoid organs of an adult animal, lymphocytes recirculate and search for invading foreign substances.

Pathogens and antigens are taken up or “captured” by antigen-presenting cells (APC) such as dendritic cells (DC). The APC serve to display peptides and antigens to the immune cells by placing these peptides on the surface of the APC in association with a major histocompatibility complex (MHC) molecule. The process of antigen capture may occur by phagocytosis of exogenous proteins or by directed transport of proteins within the cell. Alternatively, antigens may be derived from proteins synthesized within the cell. Next, antigens are processed into antigenic peptides by proteolytic degradation within the APC. The antigenic peptides are further complexed with an MHC molecule for presentation at the cell surface. Once an antigenic peptide is displayed by an MHC molecule on the APC surface, a cell-mediated immune reaction may follow which requires an interaction between the APC and a T-cell. This interaction can trigger several effector pathways, including activation of T-cells and stimulation of T-cell production of cytokines.

Interaction of an APC with a T-cell is determined by several major components. These components include:
(a) the T-cell surface marker,
(b) costimulatory molecules
(c) the class of MHC molecule; and
(d) the T-cell receptor (TCR).

T-cells can be subdivided by the presence of the surface markers CD4 and CD8. T-cells expressing CD8 are often known as suppressor or cytotoxic T-cells. T-cells expressing CD4 are often known as helper or inducer T cells. However, the CD8/CD4 dichotomy refers to the pattern of MHC association and antigen recognition. The CD8/CD4 nomenclature does not distinguish between cytotoxic and non-cytotoxic cells. The CD4 molecule binds to conserved structures of the class II MHC molecule. The CD8 molecule binds to conserved structures of class I MHC molecule. Furthermore, class I molecules are involved in processing endogenous antigens whereas class II molecules are involved in processing exogenous antigens.

The second factor important in APC/T-cell interaction is the MHC. As indicated above, the CD4 and CD8 molecules bind to the conserved structures of class II and class I molecules, respectively. Class I and class II molecules are the most polymorphic proteins known and play a major role in the immune system in the recognition of self and non-self. The heterogeneity of MHC molecule is observed at the level of haplotype or the combination of classes I and II MHC molecules encoded on a single chromosome. In the human, three distinct genetic loci designated HLA-A, HLA-B and HLA-C, have been identified encoding class I molecules. Similarly, the three distinct loci encoding class II MHC molecules include HLA-DP, HLA-DR and HLA-DQ. The multiple loci of MHC genes contribute to the complexity of self and non-self recognition process.

The third component important in APC/T-cell interactions is the T-cell receptor (TCR). The TCR is responsible for the antigenic specificity of the T-cell and may only bind antigenic peptide that is associated with the polymorphic determinants of an MHC. Because the binding of the T-cell receptor is specific for a complex comprising an antigenic peptide and the polymorphic -portion of the MHC molecule, T-cells may not respond or respond poorly when an MHC molecule of a different genetic type is encountered. This specificity of binding results in the phenomenon of MHC-restricted T-cell recognition and T-cell cytotoxicity.

In order to activate naive T-cells, the simultaneous delivery of an antigen-specific signal and a co-stimulatory signal is required. Specifically, ligation of the T-cell receptor and co-receptor does not, on its own, stimulate naïve T-cells to proliferate and differentiate into effector T-cells. The antigen-specific clonal expansion of naive T-cells requires a second, co-stimulatory signal which, in the case of CD4⁺ T cells, is delivered by the same antigen presenting cell on which the T-cell recognizes its specific antigen. Activation of CD8 T-cells also requires both signals to be presented by a single cell. Examples of two well characterized co-stimulatory molecules which are present on antigen presenting cells are the glycoproteins B7.1 and B7.2. These molecules interact with the T-cell surface receptors CD28 and CTLA-4, respectively. In the absence of co-stimulation, antigen recognition inactivates naive T-cells, inducing the state of anergy. Specifically, the anergic T-cells are unable to produce IL-2 which thereby prevents them from proliferating and differentiating into effector cells upon exposure to the MHC-antigen complex. In its immunologically normal context, anergy induction via this mechanism contributes to the induction of T-cell tolerance to self tissue antigens. However, the aberrant induction of this tolerance mechanism can lead to the onset of autoimmune conditions.

In pathogen-infected cells, proteins of the pathogens are degraded inside the cell. Some of the resulting peptides are transported into the lumen of the endoplasmic reticulum and may form complexes with class I MHC molecules. Antigenic peptides associate with the MHC molecules (Suto et al., Science 269: 1585-1588, 1995; Srivastava et al., Immunogenetics 39: 93-98, 1994) and the resulting peptide-MHC complexes are transported to and accumulate on the cell surfaces where they are recognized by receptors on T-cells (Yewdell et al., Adv. Immunol. 52: 1-123, 1992; Bevan, J. Exp. Med. 182: 639-641, 1995).

T-cells (T-lymphocytes) are the critical regulatory and effector cells of the adaptive immune system. T-cells develop and undergo selection in the thymus and then mature into functional T-cells in the tissues after receiving a series of signals. Early signals are triggered by specific antigen-MHC complexes on the surface of APC. The later signals may be provided by cytokines produced by CD4⁺ helper T-cells, such as interleukin-2 (IL-2) and interleukin-4 (IL-4), interleukin-7 (IL-7) and interleukin-12 (IL-12).

Studies with experimental animal tumors as well as spontaneous human tumors have demonstrated that many tumors express antigens that can induce an immune response. Some antigens are unique to the tumor and some are found on both tumor and normal cells. Several factors can greatly influence the immunogenicity of the tumor including, for example, the specific type of carcinogen involved and immunocompetence of the host and latency period (Old et al., Ann. N.Y. Acad. Sci. 101: 80-106, 1962; Bartlett, J. Natl. Cancer Inst. 49: 493-504, 1972). It has been demonstrated that T-cell-mediated immunity is of critical importance for rejection of virally and chemically induced tumors (Klein et al., Cancer Res. 20: 1561-1572, 1960; Tevethia et al., J. Immunol. 13: 1417-1423, 1974). The cytotoxic T-cell response is the most important host response for the control of growth of antigenic tumor cells (Anichimi et al., Immunol. Today 8: 385-389, 1987).

Adoptive immunotherapy of cancer takes the therapeutic approach, wherein immune cells with anti-tumor reactivity are administered to a tumor-bearing host, with the objective that the immune cells cause either directly or indirectly, the regression of an established tumor. Immunization of hosts bearing established tumors with tumor cells or tumor antigens has generally been ineffective since the tumor is likely to have elicited an immunosuppressive response (Greenberg, Chapter 14, in Basic and Clinical Immunology, 6^{th} ed., ed. by Stites, Stobo and Wells, Appleton and Lange, pp. 186-196, 1987). The delivery of tumor cells and tumor antigens with dendritic cells has improved the outcomes of immunization for cancer.

Animal models have been developed in which hosts bearing advanced tumors can be treated by the transfer of tumor-specific syngeneic T-cells (Mule et al., Science 225: 1487-1489, 1984). Autologous reinfusion of peripheral blood lymphocytes or tumor-infiltrating lymphocytes (TIL) have been proposed to treat several human cancers (Rosenberg, U.S. Pat. No. 4,690,914; Rosenberg et al., N. Engl. J Med. 319: 1676-1680, 1988). For example, TIL expanded in vitro in the presence of IL-2 have been adoptively transferred to cancer patients, resulting in tumor regression in select patients with metastatic melanoma. Melanoma TIL grown in IL-2 have been identified as activated T-cells CD3⁺ HLA-DR⁺, which are predominantly CD8⁺ cells with unique in vitro anti-tumor properties. Many long-term melanoma TIL cultures lyse autologous tumors in a specific class I MHC molecule and T-cell antigen receptor-dependent manner (Topalian et al., J. Immunol. 142: 3714, 1989).

Application of these methods for treatment of human cancers would entail isolating a specific set of tumor-reactive lymphocytes present in a patient, expanding these cells to large numbers in vitro and then putting these cells back into the host by multiple infusions. However, the methods of Rosenberg for generating tumor-reactive lymphocytes require the use of intact irradiated tumor cells with potential broad antigen specificity, as a source of stimulation of lymphocytes. Additionally, since T-cells expanded in the presence of IL-2 are dependent upon IL-2 for survival, infusion of IL-2 after cell transfer prolongs the survival and augments the therapeutic efficacy of cultured T-cells (Rosenberg et al., N. Engl. J. Med. 316: 889-897, 1987). However, the toxicity of the high-dose IL-2 and activated lymphocyte treatment has been considerable, including high fevers, hypotension, damage to the endothelial walls due to capillary leak syndrome and various adverse cardiac events such as arrhythmias and mycocaridal infarction (Rosenberg et al. (1988) supra). Furthermore, the demanding technical expertise required to generate TILs, the quantity of material needed and the severe adverse side effects limit the use of these techniques to specialized treatment centers.

It would be desirable, therefore, to have a method for generating a large number of activated/stimulated T-cells reactive to any antigen or a large repertoire of antigens without reliance on intact tumor cells but which has the convenience of in vitro culture. It would also be useful to determine in vitro which antigens are sufficiently immunogenic to warrant immunotherapy without the expense and time of conducting clinical trials.

In vitro priming of T-cells has been attempted with some success in the past but the results have not enabled this practice to be routinely adopted. One major problem has been the long time required to generate antigen-reactive T-cells. The conventional method (shown in FIG. 1) required over 30 days to generate cytotoxic T-cells. There is a need, therefore, to develop an improved in vitro T-cell priming protocol.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention provides a method for generating mammalian or avian T-cells specific for particular antigens. These are referred to herein inter alia as antigen-reactive T-cells. Generally, the T-cells are CD8⁺ cytotoxic T-cells which are specific for an antigen such as but not limited to a naïve or previously tolerized antigen. The responding CD8⁺ T cells may be used for adoptive immunotherapy in addition to priming the DC/CD4⁺ population, which may be injected to expand CD8⁺ T cells in vivo. The method involves first co-incubating mature APC such as dendritic cells (DC) and CD4⁺ T-cells, then exposing them to target antigen. Various activation and/or immune stimulating components may also be included such as a peptide likely to be recognized by the CD4⁺ T-cells. One such peptide is the tetanus toxoid p30 peptide. Given the widespread vaccination of human subjects against tetanus, most human subjects have CD4⁺ T-cells, which recognize p30. Tetanus toxoid p30 is an example of a cognate interactive peptide, which facilitates APC-CD4⁺ T-cell interaction. Regardless of HLA-DR haplotype, all tetanus toxoid vaccinated individuals can mount a CD4⁺ response to components of the p30 peptide. These activated p30-specific CD4⁺ T cells are then able to potently activate cognate DC, which have also been pulsed with the p30 peptide. The next stage is contacting the primed DC/CD4⁺ T-cell population with CD8⁺ T-cells. Another useful additive is an interleukin (IL) such as IL-2 or IL-7. Approximately ten days later, the bulk culture population is screened for cytotoxic T-cells specific for the target antigen. The cytotoxic T-cells may also be isolated and used in cellular immunotherapy (also referred to as CTL [cytotoxic T-lymphocyte] therapy).

Assessment of the presence of cytotoxic T-cells is by any number of means such as ELISPOT, cytokine secretion, tetramer analysis, amongst others. FACS sorting, immunoabsorption and magnetic beads may be employed to isolate specific cytotoxic T-cells.

The mammal may be a human or other primate, livestock animal, laboratory test animal, companion animal or capture wild animal. The subject may also be an avian species. Preferably, however, the subject is a human.

The method of the present invention is useful for assessing the factors affecting in vitro priming of mammalian or-avian-cytotoxic T-cell responses to any antigen such as naive or tolerized antigens. The method can, therefore, test APC populations, cellular interactions and the influences of cytokines.

Importantly, the present invention enables particular antigens to be assessed such as antigens on cancer cells, pathogen cells, viruses and cells infected by viruses. This alleviates the need for expensive and time consuming clinical trials.

The present invention provides, therefore, an in vitro T-cell priming system useful for diagnostic or potential therapeutic target applications. Furthermore, once primed, T-cells can be cloned then expanded and returned to a subject such as in autologous CTL therapy. Alternatively, vaccine preparations comprising DC/CD4+T-cell populations primed with respect to a target antigen may be generated.

The bulk CTL culture (including non-antigen specific cells) may be expanded followed by selection of antigen-specific cells and subsequent cloning of antigen-reactive CTL, or specific antigen reactive T-cells may be directly cloned from the unexpanded bulk CTL culture population.

Consequently, the present invention provides an in vitro assessment of priming with respect to an antigen, enables preparation of an in vivo cellular vaccine or enables priming in vitro to facilitate early cloning in vitro.

A list of abbreviations used herein is provided in Table 1.

TABLE 1
Abbreviations

| ABBREVIATION | DESCRIPTION |
|---|---|
| APC | antigen-presenting cells |
| CM | complete medium |
| CTL | cytotoxic T-lymphocytes |
| DC | dendritic cells |
| IL | interleukin |
| MHC | major histocompatibility complex |
| NK cells | natural killer cells |
| PBMC | peripheral blood mononuclear cells |
| polyI:C | polyinosinic polycytidylic acid |
| TRE | T-cell receptor-expressing cell |

### BRIEF DESCRIPTION OF THE FIGURES

FIG. 1 is a diagrammatic representation of a prior art method for generating cytotoxic T-cells in vitro. Generally, the method takes a minimum of 25-35 days.

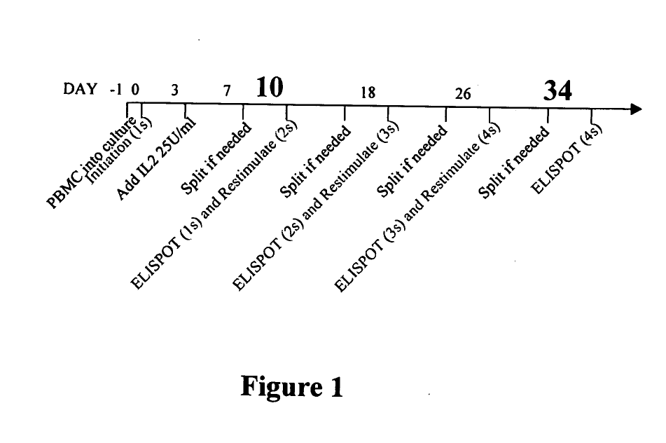

FIG. 2 is a diagrammatic representation of another version of the in vitro method of the present invention to obtain cytotoxic T-cells in approximately 10 days.

FIG. 4 is a diagrammatic representation of another version of the in vitro method of the present invention to obtain cytotoxic T-cells in approximately 10 days.

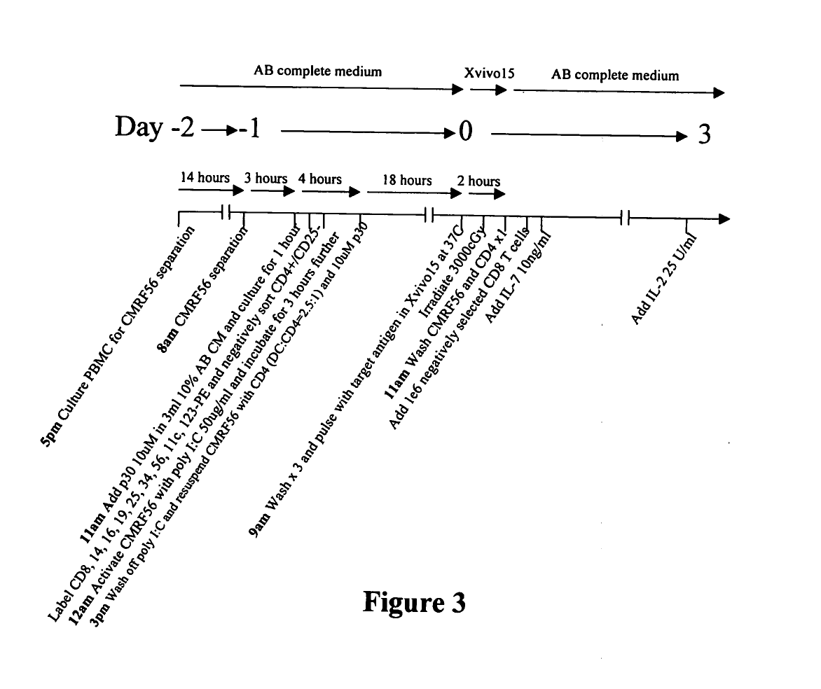

### DETAILED DESCRIPTION OF THE INVENTION

The present invention provides an in vitro T-cell priming system. Cytotoxic T-cells (also referred to herein as cytotoxic T-lymphocytes, CTLs and CD8⁺ T-cells) are generated specific to particular antigens. Such cytotoxic T-cells are also referred to as antigen-reactive T-cells.

The in vitro process involves generating a primed population of APC with respect to an antigen using CD4⁺ T-cells and then using this population to induce CD8⁺ cytotoxic T-cells specific for this antigen.

One key aspect of the present invention is selecting an APC population which is in a relatively mature state. The method of the present invention permits the generation of cytotoxic T-cells in less than 20 days and preferably in about 10 days.

Accordingly, one aspect of the present invention contemplates a method for generating a population of T-cells specific for an antigen, said method comprising isolating a population of substantially mature APC, co-incubating the substantially mature APC population with a population of CD4⁺ T-cells, a population of CD8⁺ T-cells and a target antigen for a time and under conditions sufficient to generate CD8⁺ T-cells specific for said antigen.

Reference to “co-incubation” is not intended to limit the present invention to simultaneous incubation of all three cell populations and the target antigen. Although simultaneous incubation may occur, the method extends to sequential incubation and addition of cells and/or antigens.

In another embodiment, the present invention provides a method for generating a population of T-cells specific for an antigen, said method comprising contacting a population of CD8⁺ T-cells and then screening for the presence of cytotoxic T-cells specific for said antigen.

Generally, three separate blood samples are obtained from one subject. Alternatively, tumor infiltrating lymphocytes, tumor-associated lymphocytes, bone marrow or hematopoietic cells from any part of the body may be obtained. As indicated above, the term “subject” may be a human subject or non-human subject. The three samples are used to generate the APC, CD4⁺ T-cells and CD8⁺ T-cells. One skilled in the art will immediately recognize that a single sample may be obtained from a subject, which is then split into sub-samples. The present invention extends to removing samples and maintaining these blood samples until required. In a preferred embodiment, however, separate, fresh blood samples are collected just prior to use.

In one particular embodiment, the present invention contemplates a method for generating antigen-reactive T-cells in vitro, said method comprising:
(i) obtaining a population of substantially mature APC, co-incubating said APC population with a cognate reactive peptide to generate a mature APC population expressing the cognate reactive peptide or a T-cell interacting portion thereof on the surface of the APC (referred to herein as “activated APC”);
(ii) co-incubating the activated APC with a population of CD4⁺ T-cells;
(iii) co-incubating the APC/CD4⁺ T-cell population with a target antigen;
(iv) co-incubating the APC/CD4⁺ T-cell mixture with CD8⁺ T-cells; and
(v) isolating and/or screening for the presence of cytotoxic T-cells reactive to said antigen.

Preferably, the CD4⁺ T-cells are CD4⁺ CD25⁻ T-cells.

An “antigen-presenting cell” or its abbreviations “APC” as used herein, refers to a cell or cells capable of endocytotic adsorption, processing and presenting of an antigen. The term “antigen presenting” means the display of antigen as fragments, generally peptide fragments, bound to MHC molecules, on the cell surface. Many different kinds of cells may function as APC including, for example, macrophages, B cells, follicular DC and PBMC DC and monocyte-derived DC.

PBMC DC and monocyte-derived DC are the most preferred APC of the present invention.

Accordingly, another aspect of the present invention contemplates a method for generating antigen-reactive T-cells in vitro, said method comprising:
(i) obtaining a population of substantially mature DC, co-incubating said DC population with a cognate reactive peptide to generate a mature DC population expressing the cognate reactive peptide or a T-cell interacting portion thereof on the surface of the DC (referred to herein as “activated DC”);
(ii) co-incubating the activated DC with a population of CD4⁺ T-cells;
(iii) co-incubating the DC/CD4⁺ T-cell population with a target antigen;
(iv) co-incubating the DC/CD4⁺ T-cell mixture with CD8⁺ T-cells; and
(v) isolating and/or screening for the presence of cytotoxic T-cells reactive to said antigen.

The use of numbered steps above is not to be taken as limiting the method to any one order or discrete step. It is possible that two or more steps may be combined together and/or the order changed.

An “antigen” is any organic or inorganic molecule capable of stimulating an immune response. The term “antigen” as used herein extends to any molecule such as, but not limited, to a peptide, polypeptide, protein, nucleic acid molecule, carbohydrate molecule, organic or inorganic molecule capable of stimulating an immune response. A peptide antigen is particularly preferred.

“Lymphocytes” may be T-lymphocytes or T-cells or B-lymphocytes or B-cells. Preferred lymphocytes of the present invention are cytotoxic T-cells and include T-cell-receptor-expressing cells. The term “T-cell receptor-expressing cell” or its abbreviation “TRE” refer to any thymus-derived cell capable of detecting an antigen and effecting a cell-mediated and/or a humoral immune response. Preferred TRE are T-cells. The terms “T-cell” and “T-lymphocyte” are used throughout synonymously. The present invention extends, however, to encompass embodiments wherein the responder cell is a B-lymphocyte.

Particularly useful APC in the context of the present invention are DC. DC are a population of widely distributed leukocytes that are highly specialized in antigen presentation via MHC I or MHC II antigen (e.g. peptide) complexes. As used herein, the term “dendritic cell” and DC refer to DC in their broadest context and includes any DC that is capable of antigen presentation. The term includes all DC that initiate an immune response and/or present an antigen to T-cells and/or provide T-cells with any other activation signal required for stimulation of an immune response. The most preferred DC is a DC from the PBMC.

Reference herein to “DC” should be read as including reference to cells exhibiting dendritic cell morphology, phenotype or functional activity and to mutants or variants thereof and to precursor cells of DC. The morphological features of DC may include, but are not limited to, long cytoplasmic processes or large cells with multiple fine dendrites. Phenotypic characteristics may include, but are not limited to, expression of one or more of MHC class I molecules, MHC class II molecules, CD1, CD4, CD11c, CD123, CD8α, CD205 (Dec-205), 33D1, CD40, CD80, CD86, CD83, CD45, CMRF-44, CMRF-56, CD209 (DC-SIGN), CD208 (DC-LAMP), CD207 (Langerin) or CD206 (macrophage mannose receptor). Functional activity includes, but is not limited to, a stimulatory capacity for naive allogeneic T cells. Likewise, reference herein to “T-cell” should be read as including reference to cells which express one or more T-cell-type receptor and which carry out the one or more functions associated with cells generated in the thymus and to mutants or variants thereof. “Variants” include, but are not limited to, cells exhibiting some but not all of the morphological or phenotypic features or functional activities of DC and/or T-cells. “Mutants” include, but are not limited to, DC and/or T-cells which are transgenic wherein said transgenic cells are engineered to express one or more genes such as genes encoding antigens, immune modulating agents or cytokines or receptors. Reference herein to a DC and/or T-cells refers to both partially differentiated and fully differentiated DC and/or T-cells and to activated and non-activated DC and/or T-cells.

The APC (e.g. DC) used in the method of the present invention are generally in a substantially mature state.

A reference to an APC and/or cell being “immuno-active”, or other forms thereof such as “immuno-activity”, is a reference to a range of in vivo or in vitro activities of APC and/or cells, such as occurs in the context of an immune response. For example, immune activities contemplated herein include inter alia one or more of antigen endocytosis, antigen processing and/or presentation, as well as antigen detection or recognition or effecting the lysis of target cells displaying particular antigens. In the context of the present invention, a preferred APC is a DC and a preferred lymphocyte is a T-cell. Reference hereinafter to DC includes other APC.

As detailed above, the range of immuno-activities potentially displayed by a DC encompasses and includes, inter alia, antigen endocytosis, processing and presentation, on contact with an agent capable of eliciting such a response. Similarly, the range of immuno-activities potentially displayed by a lymphocyte encompasses and includes, inter alia, activation of macrophages, stimulation of B-cells to produce antibody and causing the lysis of particular target cells displaying recognized antigens. The modulation of such “immuno-activity”, therefore, refers to the ability to alter, suppress or increase, up- or down-regulate or otherwise affect the level and/or amount of DC and/or lymphocyte immuno-activity. Preferably, the modulation results in suppression, inhibition or down-regulation of DC and/or lymphocyte immuno-activity. In this context, modulating a cell's immuno-activity also encompasses and includes affecting the viability of the said cell or cells and, in a preferred embodiment, extends to their depletion, inactivation and/or eventual apoptosis.

The DC population is preferably obtained from PBMC. However, the present invention contemplates the use of CD34 DC and/or monocyte-derived DC and/or subsets thereof. The DC are generally incubated alone or with a cognate reactive peptide for from about 7 to about 30 hours, more preferably from about 10 to about 25 hours and most preferably from about 14 hours. A cognate reactive peptide is one, which is likely to be recognized by resting CD4⁺ T-cells. Tetanus toxoid p30 is particularly useful given the widespread vaccination against tetanus. The preferred CD4⁺ T-cells are CD4⁺ CD25⁻ T-cells.

Generally, PMBC at a concentration of from about 10⁴ to about 10⁸ cells/ml of complete medium (CM) are used.

Mature DC are positively selected using a labeled antibody to a surface antigen in order to positively select cells possessing this antigen. Any of a number of antigens may be targeted by a labeled antigen for the purposes of capturing these cells. One particularly useful antigen is defined as CMRF-56 (Hock et al., Tissue Antigens 53: 320-334, 1999. However, other antigens contemplated include CD11c, CD123, CD83, CMRF-44, CD209 (DC-SIGN).

The labeled DC population (such as PBMC carrying labeled CMRF-56 DC) are then subject to cell isolation means. In one example, the labeled antibody is biotinylated and, hence, such cells are capturable using anti-biotin microbeads or other solid support. The selected antigen positive cells (e.g. CMRF-56⁺ cells) are then eluted and stored until used.

CD4⁺ T-cells are also collected from a donor. These cells are then labeled with antibodies to a range of surface antigens such as CD8, CD14, CD16, CD19, CD25, CD34, CD56, CD11c and CD123-PE. However, CD4 is not labeled. The labeled population is then negatively sorted for CD4⁺/CD25⁻ cells. The cells are then also stored until used.

CD8⁺ T-cells also from PBMC are conveniently negatively selected and isolated using any of a number of techniques or kits such as a Miltenyi CD8⁺ T-cell isolation kit.

The method in general involves incubating mature DC with a cognate reactive peptide such as tetanus toxoid p30, which is then processed and presented on the surface of the DC. The DC population is further optionally activated using an activator, which is selected depending on the subset of DC used. For example, polyinosinic polycytidylic acid (polyI:C) is particularly useful in activating CD11c DC in this regard. The choice of activator depends on the population of DC and their level of maturity. Populations of DC include monocyte-derived DC, PMBC DC and CD34 DC and/or subsets thereofIn one preferred embodiment, the DC population is exposed to tetanus toxoid p30 and polyI:C to generate a population of DC with p30 on the surface.

Activants and other non-cellular ingredients are removed by washing and the activated, mature DC population is co-incubated with CD4⁺ cells isolated and described above. Further cognate reactive peptides such as tetanus toxoid p30 may be added at this point. Incubation may be from hours to overnight if more convenient.

After washing the DC/CD4⁺ cell mixture, the resuspended cells are incubated in the presence of a target antigen, generally a target peptide.

The cell mixture is again washed and CD8⁺ T-cells added and the mixture further incubated.

Various interleukins may be added at varying stages and the cell mixture may also be irradiated. CD8⁺ cytotoxic cells are then identified and/or isolated using techniques such as ELISPOT, a cytokine secretion assay or tetramer analysis. Antigen-reactive T-cells may also be FACS sorted or isolated by immunoabsorption and/or immunomagnetic sorting.

The present invention provides, therefore, a method for priming T-cells in vitro for a target antigen, said method comprising co-incubating together or at different times mature, activated DC, CD4⁺ T-cell and CD8⁺ T-cells in the presence of said target antigen for a time and under conditions sufficient for CD8⁺ cytotoxic T-cells to generate with specificity for said antigen and then isolating said CD8⁺ T-cells.

The present invention has particular advantages in being able to obtain primed T-cells within 3-20 days as opposed to previous methods which required at least 30-40 days, depending on the antigen. Generally, the primed T-cells can be obtained in under five days or in three or four days.

A further advantage is that the instant method avoids the major costs and time of conducting clinical trials for cancer and pathogen antigens.

The present invention further contemplates a method of treatment of a subject comprising first identifying a target antigen by screening for primed T-cells reactive to said antigen by the method of co-incubating mature, activated DC, CD4⁺/CD25⁻ T-cell and CD8⁺ T-cells in the presence of said target antigen for a time and under conditions sufficient for CD8⁺ cytotoxic T-cells to generate with specificity for said antigen and then isolating said CD8⁺ T-cells and then generating a vaccine based on an antigen to which T-cells are capable of being primed in vitro.

Alternatively, the in vitro primed cytotoxic T-cells can be cloned and expanded and then returned to the subject. This is referred to herein as CTL therapy or adoptive therapy or autologous immunotherapy.

Still another alternative is to isolate DC/CD4⁺ T-cells primed for a particular antigen and then administer the cell population to a subject. This facilitates early cloning.

Preferably, the subject is a human. However, the present invention extends to other primates, livestock animals (e.g. sheep, horses, cows, pigs, donkeys), laboratory test animals (e.g. mice, rats, rabbits, guinea pigs, hamsters), companion animals (e.g. dogs, cats) and captured wild animals as well as avian species.

According to the present invention, adoptive therapy is carried out by obtaining in vitro antigen-primed T-cells, inducing clonal expression in cell culture of the cytotoxic T-cells and then administering the antigen-reactive T-cells into the subject. When infused into the subject, antigen-reactive T-cells of the present invention can specifically target and/or directly kill target cells or viruses in vivo that bear the same antigen as the antigenic cells, thereby inhibiting cancer growth or preventing or limiting the spread of the pathogen in the recipient. For adoptive therapy, antigen-reactive T-cells would preferably be purified and/or enriched.

In a preferred embodiment of the invention, the DC and T-cells and the recipient of the antigen-reactive T-cells or DC/CD4⁺ T-cells have the same MHC haplotype. In a preferred embodiment, the present invention is directed to the use of autologous T-cells or DC/CD4⁺ T-cells stimulated in vitro with autologously-derived antigen for the treatment or prevention of cancer or infectious disease in the same subject from which the T-cells (or preferably, all the immune cells) and antigen were originally derived. In a more preferred aspect, the immune cells and antigenic cells are isolated from a human subject in need of cellular immunotherapy.

In another embodiment of the subject invention, the T-cells and/or DC and the recipient have the same haplotype while the antigenic cells are allogeneic to the T-cells or DC and the recipient but matched with at least one MHC allele, i.e. antigenic cells are used to activate T-cells, which T-cells or DC/T-cells are then administered to a recipient from which the T-cells and/or DC were originally obtained, and in which the antigenic cells and the T-cells and/or DC share at least one but not all MHC alleles.

In a least preferred embodiment of the present invention, the antigenic cells, the T-cells and/or DC and the recipient are all allogeneic with respect to each other but all have at least one common MHC allele shared among the antigenic cells, the T-cells and/or DC and the recipient.

According to a specific embodiment of the present invention, antigen-reactive CD8⁺ T-cells are generated and used prophylactically to prevent infection or development or remission of cancer. Alternatively, a population of CD4⁺ T-cells are generated primed for a target antigen. In another embodiment, such T-cells can be used therapeutically to treat infection or its sequelae, or to treat cancer. Preferably, the antigenic cells used to generate the antigen-reactive T-cells or primed DC are syngeneic to the subject to which they are to be administered, e.g. are obtained from the subject. However, if cancer cells or pathogen-infected cells that are syngeneic to the subject are not available for use, the methods of the present invention provide that such antigenic cells, which have the same MHC haplotype as the intended recipient of the cells can be prepared in vitro using non-cancerous or uninfected cells (e.g. normal cells) collected from the recipient. For example, depending on the mode of transmission of the pathogen, normal cells obtained from the recipient can be infected in vitro by incubation with the pathogen or other pathogen-infected cells and then used to prime the host immune cells in vivo. In another embodiment, lysates or preparations of cells infected with a pathogen in vitro or thereof can be used to pulse DC or primed immune cells comprising DC in vitro. In still another embodiment, lysates or preparations of cells infected with a pathogen in vitro can be used for restimulation of antigen-reactive T-cells of the subject invention.

In another embodiment, normal cells can be induced to become cancerous or transformed, e.g. by treatment with carcinogens, such as chemicals and/or radiation or infection with a transforming virus and then used for priming directly. In another embodiment, lysates or preparations of such cancerous, transformed or infected cells can be used to pulse immune cells or DC in vitro. In still another embodiment, the lysates or preparations of such cells can be used for restimulation of the antigen-reactive T-cells of the present invention.

Furthermore, in another embodiment, if the cloned gene of the antigen of interest is available, normal cells from the subject can be transformed or transfected with the gene such that the antigen of interest is expressed recombinantly in the cells and then such cells can be used in the priming, pulsing and/or restimulation reactions. In a less preferred aspect, antigenic cells for use can be prepared from cells that are not syngeneic but that have at least one MHC allele in common with the intended recipient.

By following the present methods, any antigenic cells of interest can be used to prime T-cells or DC in vitro, even cancer cells or infected cells that are considered unsafe for use in active immunization. Such primed T-cells are then exposed to DC pulsed with a cognate reactive protein.

There are many advantages of immunotherapy as provided by the present invention. Tumor bulk is minimal following surgery and immunotherapy is most effective in this situation. In a specific embodiment, the preventive and therapeutic methods of the invention are directed at enhancing the immunocompetence of a cancer patient either before surgery or after surgery and enhancing cell-mediated tumor-specific immunity against cancer cells with the objective being inhibition of proliferation of cancer cells and total eradication of residual cancer cells in the body.

In another preferred aspect, in which antigen-reactive T-cells or DC/CD4⁺ T-cells reactive against human cancer cells can be used, alone or in conjunction with surgery, chemotherapy, radiation or other anti-cancer therapies, to eradicate metastases or micrometastases, or inhibit the growth of metastases or micrometastases, the antigen-reactive T-cells or DC/T-cells provided by the present invention are administered in vivo, to the subject having or suspected of having the metastases or micrometastases. For example, to purge bone marrow of cancer cells during bone marrow transplantation, bone marrow from the donor is contacted in vitro with the antigen-reactive T-cells or DC/T-cells provided by the subject invention, so that antigen reactive T-cells lyse any residual cancer cells in the bone marrow, prior to administering the bone marrow to the subject for purposes of hematopoietic reconstitution. The bone marrow transplantation is preferably autologous.

Moreover, if cancer patients undergo surgery with anesthesia and subsequent chemotherapy, the resulting immunosuppression experienced by the patient may be lessened by cellular immunotherapy in the pre-operative period, thereby reducing the incidence of infectious complications. There is also the possibility that tumor cells are shed into circulation at surgery and, thus, effective immunotherapy applied at this time can eliminate these cells in vivo. The present invention thus provides a method of prophylaxis or treatment comprising administering to a cancer patient the antigen-reactive T-cells provided by the present invention, reactive against an antigen of the patient's cancer cells, prior to, during and/or subsequent to surgery and/or chemotherapy undergone by the cancer patient.

In a preferred aspect involving acute viral infection of humans, CD8⁺ cells are reactive against virus-infected cells of a human subject and can be rapidly generated and reinfused back to the subject for controlling the viral infection.

In another preferred aspect, the present invention provides CD8⁺ T-cells reactive against an opportunistic pathogen that infects immunosuppressed or immunodeficient subjects, such as but not limited to cytomegalovirus, Toxoplasma gondii, Herpes zoster, Herpex simplex, Pneumocystis carinii, Mycobacterium avium-intracellulare, Mycobacterium tuberculosis, Cryptosporidium and Candida species. The antigen-reactive T-cells of the present invention can be used therapeutically and preferably autologously, in human patients suffering from acquired immunodeficiency syndrome (AIDS) and associated infections and cancers, or prophylactically in subjects that are infected with the human immunodeficiency virus (HIV) or HIV seropositive subjects or otherwise at high risk for developing AIDS.

Antigen-reactive T-cells are generated in vitro by stimulation and proliferation of a subset of T-cells according to the methods herein described. After sufficient time is given for the in vitro stimulation reaction to occur, the T-cells can be tested for proliferation, cytotoxicity, cytokine secretion. Alternately, cells may be restimulated to enhance or sustain the proliferation or stored or maintained in long-term culture for later use. The method of the present invention is also useful in identifying potentially useful antigens in a subsequent immunotherapy program. This reduces the need for multiple clinical trials.

Any antigenic cell, e.g. cancer or infected cells, may be used in the present methods. The source of the antigenic cells may be selected, depending on the nature of the disease with which the antigen is associated, and the intended use of the resulting antigen-reactive T-cells. In one embodiment of the subject invention, any tissues or cells isolated from a cancer, including cancer that has metastasized to multiple sites, can be used in the present method. In another embodiment of the invention, any cell that is infected with a pathogen in particular an intracellular pathogen such as a virus bacterium, fungus, parasite or protozoan, can be used. Typically, by way of example but not limitation, cancer cells can be isolated from a tumor that is surgically removed from a human patient who will be the recipient of the antigen-reactive T-cells or other immunotherapeutic agent, such as a cytotoxic antibody. Prior to use, solid cancer tissue, pathogen-infected tissue or aggregated cancer cells should be dispersed, preferably mechanically, into a single cell suspension by standard techniques. Enzymes such as but not limited to collagenase and Dnase may also be used to disperse cancer cells. Typically, approximately 2-3 million antigenic cells are used per priming reaction in the method. Thus, if necessary, the cancer or infected cells may be cultured by standard techniques under growth conditions in vitro to obtain the desired number of cells prior to use. Primary tissue or cell lines can also be used.

After the in vitro stimulation/restimulation reaction, the mixed cell culture comprising responding T-cells including the antigen-reactive T-cells of the present invention are assayed for reactivity using a variety of assays such as ELISPOT, ⁵¹Cr release assay, a cytokine assay or any assay known in the art for measuring reactivity of immune effector cells.

Alternatively, the reactivity of the responding T-cells can also be determined by measuring the levels of cytokines, such as but not limited to, tumor necrosis factor (TNF), interferon gamma (IFNγ) granulocyte-macrophage colony stimulating factor (GM-CSF), IL-2, IL-4, IL-10, IL-12 and IL-5 secreted upon stimulation or restimulation. Proliferation of T-cells may also be examined by standard methods in the art, such as ³H-thymidine incorporation, FACS analysis, growth curves and cytokine secretion.

The antigen-reactive T-cells or a DC/CD4⁺ T-cell population of the present invention may be infused into a recipient systemically, preferably intravenously. Recipients generally receive from about 10⁵ to 10¹¹ purified or enriched cells (or a composition comprising the same) per administration and preferably about 10⁶ to about 10⁸ immune cells per administration or about 10⁵ to about 10⁷ purified or enriched cells (or a composition comprising the same) per administration, depending on the condition of the patient. Preferably, such cells are administered to an autologous recipient.

Various delivery systems are known and can be used to administer the cells of the present invention, e.g. encapsulation in liposomes, microparticles, microcapsules. Methods of introduction include but are not limited to intradermal, intramuscular, intraperitoneal, intravenous, subcutaneous, intranasal and oral routes. The cells may be administered by any convenient route, for example, by infusion, and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter.

In a specific embodiment, it may be desirable to administer the pharmaceutical compositions of the present invention locally to the area in need of treatment; this may be achieved by, for example, and not by way of limitation, local infusion during surgery, by injection, by means of a catheter, or by means of an implant, said implant being of a porous, non-porous or gelatinous material, including membranes such as silastic membranes or fibers. In one embodiment, administration can be by direct injection at the site (or former site) of a cancer or infection or directly into the cancer or tumor.

The present invention also provides pharmaceutical compositions. Such compositions comprise a therapeutically or prophylactically effective amount of antigen-reactive T-cells or a DC/CD4⁺ T-cell population of the present invention and a pharmaceutically acceptable carrier or excipient. Such a carrier includes but is not limited to saline, culture medium with or without serum, buffered saline, dextrose, water, glycerol, ethanol and combinations thereof. The carrier and composition can be sterile. The formulation should suit the mode of administration. The composition, if desired, can also contain minor amounts of wetting or emulsifying agents, or pH buffering agents. In a preferred embodiment, the pharmaceutical composition comprises a majority of CD8⁺ antigen-reactive T-cells.

In a preferred embodiment, the composition is formulated in accordance with routine procedures as a pharmaceutical composition adapted for intravenous administration to human beings. Typically, carriers for intravenous administration are sterile isotonic aqueous buffers. Where necessary, the composition may also include a local anesthetic such as lignocaine to ease pain at the site of the injection. Where the composition is to be administered by infusion, it can be dispensed with an infusion bottle containing sterile pharmaceutical grade water, saline or culture medium. Where the composition is administered by injection, an ampoule of sterile water or saline or culture medium for injection can be provided so that the ingredients may be mixed prior to administration.

The amount of cells of the present invention which will be effective in the treatment of a particular disorder or condition will depend on the nature of the disorder or condition and can be determined by standard clinical techniques. In addition, in vitro assays may optionally be employed to help identify optimal dosage ranges. The precise dose to be employed in the formulation will also depend on the route of administration and the seriousness of the disease or disorder and should be decided according to the judgment of the practitioner and each patient's circumstances.

The present invention also provides a pharmaceutical pack or kit comprising one or more containers filled with one or more of the ingredients of the pharmaceutical compositions of the subject invention. In particular, the subject invention contemplates a kit in compartmental form having compartments adapted to receive cells or contain reagents used in the in vitro protocol. Compartments may also be adapted for use in a FACS machine. In addition, instructions for use may also be included. The kit may be useful for clinical investigations or for research purposes.

Infectious diseases that can be treated or prevented by antigen-reactive T-cells or DC/CD4⁺ T-cell populations of the present invention are caused by infectious agents including but not limited to viruses, bacteria, fungi, protozoans and parasites.

Viral diseases that can be treated or prevented by the methods and compositions of the present invention include but are not limited to those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV-I) and human immunodeficiency virus type II (HIV-II).

Bacterial disease that can be treated or prevented by the methods and compositions of the present invention are caused by bacteria including but not limited to Mycobacterium, Rickettsia, Mycoplasma, Neisseria and Legionella.

Protozoal diseases that can be treated or prevented by the methods and compositions of the present invention are caused by protozoa including but not limited to Leishmania, Coccidioidomycoses and Trypanosoma.

Parasitic diseases that can be treated or prevented by the methods and compositions of the present invention are caused by parasites including but not limited to Chlamydia and Rickettsia.

Cancers that can be treated or prevented by antigen-reactive T-cells and methods of the present invention include but are not limited to human sarcomas and carcinomas, e.g. fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcimona, basal cell carcinoma, adenocarcinoma, seat gland carcinoma, sebaceous gland carcinoma, papillary carcinoma, paillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g. acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia and heavy chain disease.

The present invention is further described by the following non-limiting Example.

### EXAMPLE

### In Vitro Generation of Human Antigen-Specific Cytotoxic T-Cells

The following method is used for the in vitro generation of human cytotoxic T-cells specific for either naive or previously tolerized self-tumor peptide antigens. It involves isolation of DC, followed by pre-activation of the isolated DC by incubation for 4 hours with poly I:C and tetanus toxoid p30 peptide and a further 18 hours with inter alia cognate p30-specific CD4⁺/CD25⁻ T cells. Once activated, negatively selected CD8⁺ T-cells are added to the DC/CD4 mix. IL-7 is added on day 0, followed by IL-2 on day 3. If necessary, the cultures are split and IL2 is replaced on day 6 or 7, at least 3 full days before restimulation or assessment on day 10. Assessment is by ELISPOT, cytokine secretion or tetramer analysis.

In principle, this method is adaptable to any application involving the assessment of factors affecting in vitro priming of human cytotoxic T-cell responses to any antigen including naive or tolerized self-tumor antigens such as APC preparations, cellular interactions, cytokine influences and the like.

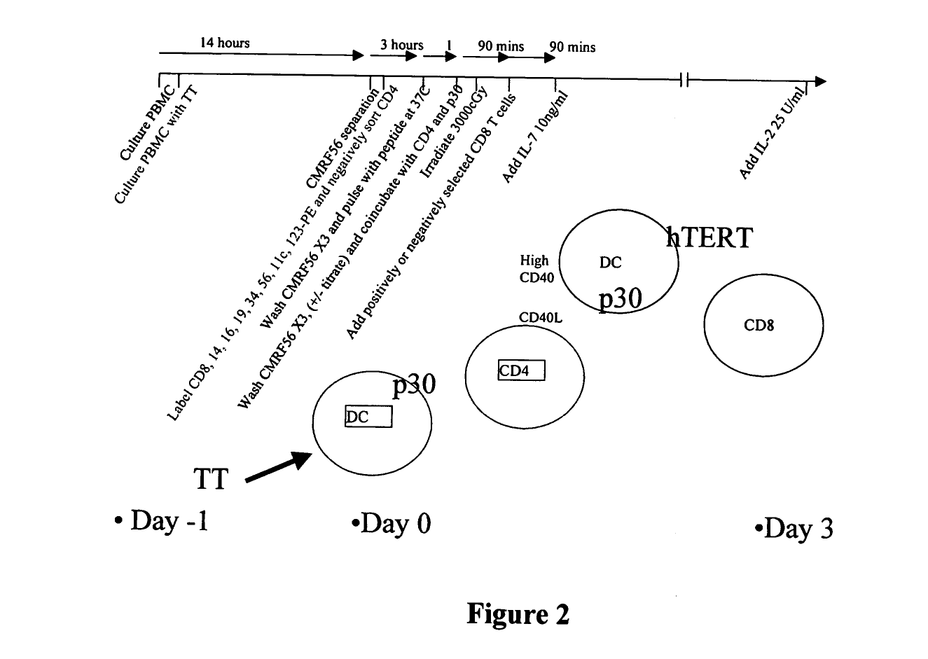

Various versions of the protocol are shown in FIGS. 2 and 3.

The following reagents were used:
1. RPMI 1640—Gibco Life Technologies Cat No 21870-076.
2. 10% AB complete medium (CM)—RPMI 1640, pooled AB serum 10%, glutamine 2 mM (Gibco Life Technologies Cat No 25030-081), MEM sodium pyruvate 1 mM (Gibco Life Technologies Cat No 11360-070), MEM non-essential amino acids 0.1 mM (Gibco Life Technologies Cat No 11140-050), HEPES buffer 10 mM (Gibco Life Technologies Cat No 15630-080), 2-mercaptoethanol 50 uM (Sigma Cat No M-7522).
3. Xvivo 15 serum free medium—BioWhittaker Cat No 04418-Q.
4. Anti-biotin magnetic beads—Miltenyi Biotec Cat No 130-090-485.
5. CD8⁺ T cell isolation kit (contains hapten-modified CD4, 11b, 16, 19, 36, 56)—Miltenyi Biotec Cat No 130-053-201; anti-hapten magnetic beads.
6. Polyinosinic polycytidylic acid (poly I:C)—Sigma Cat No P1530.
7. Tetanus toxoid p30 peptide (tt947-967)—FNNFTVSFWLRVPKVSASHLE (SEQ ID NO: 1)
8. Interleukin 7 (IL-7) - Sigma Cat No I 5896.
9. Interleukin 2 (IL-2) - Roche Cat No 1035-0490.
10. Antibodies:
   (a) Biotinylated CMRF-56 antibody (see U.S. Pat. No. 6,479,247)
   (b) Becton Dickinson—PE conjugated
      (i) CD8—Cat No 340046
      (ii) CD14—Cat No 347497
      (iii) CD16—Cat No 347617
      (iv) CD19—Cat No 340364
      (v) CD25—Cat No 347647
      (vi) CD34—Cat No 348057
      (vii) CD56—Cat No 340363
      (viii) CD11c—Cat No 347637
      (ix) CD123—Cat No 340545
   (c) Becton Dickinson—FITC conjugated
      (i) CD4—Cat No 340133
      (ii) CD8—Cat No 347313
   (d) Dako
      (i) Streptavidin-RPE-Cy5—Cat No C0050

The following method steps are follows:

1. Generation of CMRF56—DC
(a) PBMC are cultured for 14 hours at a concentration of 10-15×10⁶/ml in condition medium (CM) in non-tissue culture grade Petri dishes.
(b) Each plate is washed with 50 ml cold PBS, pool PBMC and washed once more.
(c) The pellet of cells is resuspended to a concentration of 66×10⁶/ml in cold sterile PBS/2% HS/2 mM EDTA buffer.
(d) 10 ug/mI biotinylated CMRF56 antibody is added and incubated on ice for 15-20 minutes.
(e) The resulting cells are washed with cold PBS at 10-20× the labeling volume and then resuspended to a concentration of 100×10⁶/ml in cold sterile PBS/2% HS/2 mM EDTA buffer.
(f) A small sample is taken (“before”) for FACS analysis (see below).
(g) Miltenyi anti-biotin microbeads are added at a concentration of 20 ul per 1×10⁷ PBMC and incubated for 15-20 minutes at 4° C.
(h) The beads are washed with cold PBS at 10-20× the labeling volume and resuspended to 200×10⁶/ml in sterile cold degassed PBS/2% HS/2 mM EDTA buffer.
(i) The sample is then run on Miltenyi LS (3 ml prime/sample/4×3 ml wash/5 ml elution) or MS (2 ml prime/sample/5 ml wash/2 ml elution) columns, depending on the number of cells loaded onto the column and the expected yield.
(j) The positive fraction is counted by trypan blue microscopy.
(k) Samples are stained (“before”, “negative”, “positive”) with CD14-PE, CD19-PE and streptavidin-PC5 at 1:30 in PBS/2% HS/2 mM EDTA buffer and kept on ice until read.
(l) The remaining CMRF-56 positive fraction is spun down and resuspend in 3 ml CM.

2. Generation of CD4⁺/CD25⁻ T-Cells
(a) PBMC are isolated (expect roughly 10-40% of the PBMC to be CD4⁺/CD25⁻) and washed once in cold PBS and resuspended to 2×10⁶ PBMC per 30 ul PBS/2% HS/2 mM EDTA buffer.
(b) The cells are labeled with Becton Dickinson anti CD8, 14, 16, 19, 25, 34, 56, 11c, 123-PE 1:30 and incubated on ice for 30 minutes.
(c) The labeled cells are washed with PBS/2% HS/2 mM EDTA buffer at 10-20× the labeling volume.
(d) The washed cells are resuspended in PBS/2% HS/2 mM EDTA buffer at a concentration of 5×10⁶/ml and sorted for a PE-negative population. The viable cellular yield is roughly 50% of the counted events on the FACSVantage.
(e) CD4⁺/CD25⁻ cells are counted with trypan blue.
(f) A small sample is taken for FACS analysis with Becton Dickinson CD4-FITC 1:30.
(g) The sample is spun down and resuspend in 3 ml CM. The concentration required depends on the number of DC isolated—generally, a DC:CD4 ratio of 2.5:1 is preferable.
(h) The cells are kept on ice until used.

3. Generation of Negatively-Selected Responder CD8⁺ T Cells
(a) PBMC are isolated (expect a CD8⁺ yield of roughly 10-30% of the PBMC) and washed once with cold PBS.
(b) Cells are resuspended to a concentration of 1×10⁷ PBMC per 80 ul sterile cold PBS/2% HS/2 mM EDTA buffer.
(c) 20 ul Miltenyi CD8⁺ T cell isolation kit hapten-antibody cocktail is added per 1×10⁷ PBMC.
(d) Cells are incubated at 4C for 10 minutes.
(e) Cells are washed twice with cold PBS at 10-20× the labeling volume.
(f) Cells are resuspended to a concentration of 1×10⁷ PBMC per 80 ul sterile cold PBS/2% HS/2 mM EDTA buffer.
(g) 20 ul Miltenyi CD8⁺ T cell isolation kit anti-hapten microbeads are added per 1×10⁷ PBMC.
(h) Cells are incubated at 4° C. for 15 minutes.
(i) Cells are washed twice with cold PBS at 10-20× the labeling volume and then resuspended to 200×10⁶/ml in sterile cold degassed PBS/2% HS/2 mM EDTA buffer.
(l) Cells are run on Miltenyi LS or MS columns (as above), depending on the number of cells loaded and the expected yield and the wash fraction collected (this will contain the CD8⁺ cells).
(k) Cells are counted using trypan blue.
(l) A small sample is taken for FACS analysis with Becton Dickinson CD8-FITC 1:30.
(m) CD8⁺ are spun fraction down and resuspend to a concentration of 2×10⁶/ml in CM and kept on ice until used.

The in vitro T-cell stimulation protocol is as follows:
1. Antigen presenting cells (APC) are suspended in 3 ml CM.
2. 10 uM tetanus toxoid p30 peptide is added to the cell mix and incubated at 37° C./5% CO₂ for one hour.
3. Poly I:C 50 ug/ml is then added to the mixture and is incubated for three hours at 37° C./5% CO₂.
4. Cells are thoroughly resuspended and then poly I:C washed off with 10 ml RPMI 1640.
5. Cells are resuspended with the CD4⁺/CD25⁻ T cells to a DC:CD4 ratio of 2.5:1 and a final volume of 3 ml CM.
6. Tetanus toxoid p30 10 uM is added.
7. Cells are incubated overnight (18 hours) at 37° C./5% CO₂.
8. Cells are washed x3 with 10 ml RPMI 1640.
9. Cells are resuspended in Xvivo 15 with 1 ug/ml of the target peptide.
10. Cells are then incubated for 2 hours at 37° C./5% CO₂; during this time, the cells are irradiated (3000 cGy) [the APC/CD4 population].
11. Cells are washed once with 10 ml RPMI 1640 and resuspend to a final volume of 0.5 ml CM (or 1 ml if APC dilutions are planned - make APC dilutions of 1:2 in a 48 well tissue culture plate in a final volume of 0.5 ml CM per well).
12. 1×10⁶ CD8⁺ T cells in 0.5 ml CM are added per well.
13. IL-7 10 ng/ml is added to each well.
14. On day 3, IL-2 is added to a final concentration of 25 U/ml in 50 ul CM to each well.
15. On day 6 or 7, if necessary, the cultures are split (medium not discarded) and supplemented to a total volume of 1 ml with CM and IL-2 at a final concentration of 25 U/ml.
16. Antigen-specific cytotoxic T-cells are assessed by ELISPOT, cytokine secretion assay or tetramer analysis on day 10.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Anichimi et al., Immunol. Today 8: 385-389, 1987.

Bartlett, J. Natl. Cancer Inst. 49: 493-504, 1972.

Bevan, J. Exp. Med. 182: 639-641, 1995.

Greenberg, Chapter 14, in Basic and Clinical Immunology, 6^{th} ed., ed. by Stites, Stobo and Wells, Appleton and Lange, pp. 186-196, 1987.

Hock et al., Tissue Antigens 53: 320-334, 1999

Klein et al., Cancer Res. 20: 1561-1572, 1960.

Mule et al., Science 225: 1487-1489, 1984.

Old et al., Ann. N.Y. Acad. Sci. 101: 80-106, 1962.

Rosenberg, U.S. Pat. No. 4,690,914.

Rosenberg et al., N. Engl. J Med. 319: 1676-1680, 1988.

Rosenberg et al., N. Engl. J Med. 316: 889-897, 1987

Srivastava et al., Immunogenetics 39: 93-98, 1994.

Suto et al., Science 269: 1585-1588, 1995.

Tevethia et al., J. Immunol. 13:1417-1423, 1974.

Topalian et al., J. Immunol. 142: 3714, 1989.

Yewdell et al., Adv. Immunol. 52. 1-123, 1992.

1. A method of generating a population of T-cells specific for an antigen, said method comprising
isolating a population of substantially mature antigen presenting cells (APC); and
co-incubating the substantially mature APC population with a population of CD4+ T-cells, a population of CD8+ T-cells and a target antigen for a time and under conditions sufficient to generate CD8+ T-cells specific for said antigen.

2. The method of claim 1 wherein the co-incubation comprises simultaneously admixing two or more of the mature APC, the CD4+ T-cells, the CD8+ T-cells and the target antigen.

3. The method of claim 1 wherein the co-incubation comprises the sequential addition of one or more of the mature APC, the CD4+ T-cells, the CD8+ T-cells and the target antigen in any order.

4. The method of claim 1 wherein the mature APC are co-incubated with a cognate reactive antigen to generate a mature APC population expressing the cognate reactive antigen or a T-cell interacting portion thereof (activated APC).

5. The method of claim 4 wherein the activated APC are co-incubated with a population of CD4+ T-cells.

6. The method of claim 5 wherein the activated APC/CD4+ T-cells are co- incubated with target antigen.

7. The method of claim 6 wherein the activated APC/CD4+ T-cells/antigen mixture is co-incubated with CD8+ T-cells.

8. The method of claim 7 wherein cytotoxic T-cells are selected from the CD8+ T-cells.

9. The method of claim 5 wherein the CD4+ T-cells are CD4+ CD25⁻ T-cells.

10. The method of claim 1 wherein the APC are dendritic cells (DC).

11. The method of claim 10 wherein the DC are from peripheral blood.

12. The method of claim 10 wherein the DC express one or more molecules selected from the group consisting of MHC Class I molecules, MHC Class II molecules, CDT, CD4, CD11c, CD123, CD8a, CD205, 33D1, CD40, CD80, CD86, CD83, CD45, CMRF-44, CMRF-56, CD-209, CD208, CD207 and CD206.

13. The method of claim 1 wherein the antigen is selected from the group consisting of: a peptide, polypeptide, protein, nucleic acid molecule, carbohydrate molecule, organic molecule, and inorganic molecule.

14. The method of claim 11 wherein the antigen is a peptide.

15. A method for priming T-cells in vitro for a target antigen, said method comprising:
co-incubating together or at different times, mature activated DC, CD4+ T-cells and CD8+ T-cells in the presence of said target antigen for a time and under conditions sufficient for CD8+ cytotoxic T-cells to generate with specificity for said antigen; and
isolating said CD8+ T-cells.

16. The method of claim 15 wherein primed T-cells are generated in from about three to about 20 days.

17. A method of treatment of a subject comprising:
identifying a target antigen by screening for primed T-cells reactive to said antigen by the method of co-incubating mature, activated DC, CD4+/CD25− T-cell and CD8+ T-cells in the presence of said target antigen for a time and under conditions sufficient for CD8+ cytotoxic T-cells to generate with specificity for said antigen;
isolating said CD8+ T-cells;
generating a vaccine based on an antigen to which T-cells are capable of being primed in vitro; and
administering said vaccine to said subject in an amount effective to treat said subject.

18. (canceled)

19. (canceled)

20. The method of claim 17 wherein the subject in a human, livestock animal, laboratory test animal, a captured wild animal or an avian species.

21. The method of claim 20 wherein the subject is a human.

22. The method of any one of claims 17, 29 or 30, wherein the subject is treated for a condition selected from the group consisting of hepatitis type A, hepatitis type B, hepatitis type C, influenza, varicella, adenovirus, herpes simplex type I (HSV-I), herpes simplex type II (HSV-II) , rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV-I) and human immunodeficiency virus type II (HIV-II).

23. The method of any one of claims 17, 29 or 30, wherein the subject is treated for an infection selected from the group consisting of by Mycobacterium, Rickettsia, Mycoplasma, Neisseria and Legionella.

24. The method of any one of claims 17, 29 or 30, wherein the subject is treated for an infection selected from the group consisting of Leishmania, Coccidioidomycoses and Trypanosoma.

25. The method of any one of claims 17, 29 or 30, wherein the subject is treated for an infection selected from the group consisting of Chlamydia and Rickettsia.

26. The method of any one of claims 17, 29 or 30, wherein the subject is treated for a condition selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcimona, basal cell carcinoma, adenocarcinoma, seat gland carcinoma, sebaceous gland carcinoma, papillary carcinoma, paillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g. acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia and heavy chain disease.

27. (canceled)

28. A pharmaceutical composition comprising the T-cells of claim 1 or 15.

29. A method of treatment of a subject comprising:
identifying a target antigen by screening for primed T-cells reactive to said antigen by the method of co-incubating mature, activated DC, CD4+/CD25− T-cell and CD8+ T-cells in the presence of said target antigen for a time and under conditions sufficient for CD8+ cytotoxic T-cells to generate with specificity for said antigen;
isolating said CD8+ T-cells;
cloning and expanding the in vitro primed cytotoxic T-cells; and
administering said T-cells to a subject.

30. A method of treatment of a subject comprising:
identifying a target antigen by screening for primed T-cells reactive to said antigen by the method of co-incubating mature, activated DC, CD4+/CD25− T-cell and CD8+ T-cells in the presence of said target antigen for a time and under conditions sufficient for CD8+ cytotoxic T-cells to generate with specificity for said antigen;
isolating DC/CD4+ T-cells primed for a particular antigen; and
administering said DC/CD4+ T-cells in an amount effective to treat said subject.
